(19) United States
(12) Patent Application Publication
(10) Pub. No.: US 2024/0408397 A1
(43) Pub. Date: Dec. 12, 2024

(54) SYSTEMS AND METHODS FOR SPECTRALLY BANDED NEUROMODULATION

(71) Applicant: Boston Scientific Neuromodulation Corporation, Valencia, CA (US)

(72) Inventors: Rosana Esteller, Santa Clarita, CA (US); Tianhe Zhang, Studio City, CA (US); Rafael Carbunaru, Valley Village, CA (US)

(21) Appl. No.: 18/732,221

(22) Filed: Jun. 3, 2024

Related U.S. Application Data
(60) Provisional application No. 63/471,407, filed on Jun. 6, 2023.

(51) Int. Cl. A61N 1/36; A61N 1/02; A61N 1/372

(52) U.S. Cl. CPC A61N 1/36185; A61N 1/025; A61N 1/36071; A61N 1/36132; A61N 1/37247

(57) ABSTRACT

A system may include a plurality of electrodes and a neural modulation device configured to deliver energy using at least some of the plurality of electrodes to modulate the volume of neural tissue. The neural modulation device may be configured to deliver the energy according to a modulation parameter set. The system may include a programming system configured to program the neural modulation device with the modulation parameter set for use to deliver the energy. The energy corresponds to a broad-spectrum signal having a plurality of frequency ranges, and the programming system is configured to receive user input for targeting energy to a volume of tissue, determine a stimulation configuration, including the modulation parameter set, based on the user input, and deliver the energy corresponding to the broad-spectrum signal using the plurality of electrodes according to the determined stimulation configuration.

### CLAIM OF PRIORITY

This application claims the benefit of U.S. Provisional Application No. 63/471,407 filed on Jun. 6, 2023, which is hereby incorporated by reference in its entirety.

### TECHNICAL FIELD

This document relates generally to medical systems, and more particularly, but not by way of limitation, to systems, devices, and methods applying and controlling broad-spectrum signals to structures in the nervous system.

### BACKGROUND

Neural modulation has been proposed as a therapy for a number of conditions. Often, neural modulation and neural stimulation may be used interchangeably to describe excitatory stimulation that causes action potentials as well as inhibitory and other effects. Examples of neuromodulation include Spinal Cord Stimulation (SCS), Deep Brain Stimulation (DBS), Peripheral Nerve Stimulation (PNS), and Functional Electrical Stimulation (FES). SCS, by way of example and not limitation, has been used to treat chronic pain syndromes. SCS has conventionally used as a pain therapy, but may be implemented for other therapies as well.

Broad-spectrum and/or frequency band specific “noise” stimulation has been explored previously as a way to modulate physiological function. Neuromodulation patterns (time-varying stimulation) been proposed to be applied to different stimulation parameters such as amplitude, pulse width, frequency, duty cycle, charge per second, charge per phase, cycling. These time-varying modulation signals may be created using a signal modulating waveform (e.g., random, sinewave, triangular, sawtooth, or other shape). The concept of random has similarities to noise, but there are different categories of noise, including white noise, and many different color noise (brown, pink, blue, etc.) depending on the frequency spectrum of the noise signal and on the data distribution followed by the specific type of noise.

It is desired to improve the use of broad-spectrum signals in a neuromodulation therapies.

### SUMMARY

An example (e.g., Example 1) of a system may include a plurality of electrodes on at least one lead configured to be operationally positioned for use in modulating a volume of neural tissue. The system may include a neural modulation device configured to deliver energy using at least some of the plurality of electrodes to modulate the volume of neural tissue. The neural modulation device may be configured to deliver the energy according to a modulation parameter set. The system may include a programming system configured to program the neural modulation device with the modulation parameter set for use to deliver the energy. The energy corresponds to a broad-spectrum signal having a plurality of frequency ranges, and the programming system is configured to receive user input for targeting energy to a volume of tissue, determine a stimulation configuration, including the modulation parameter set, based on the user input, and deliver the energy corresponding to the broad-spectrum signal using the plurality of electrodes according to the determined stimulation configuration. By way of example, a stimulation configuration may include a modulation parameter set which may include amplitude, pulse width, and frequency, an electrode configuration which may include fractionalization and polarity, and pulse timing which may include a time delay and phase offset.

In Example 2, the subject matter of Example 1 may optionally be configured such that a first user input defines a target type for the volume of tissue and a direction of a modulation field generated by the delivered energy, and the stimulation configuration is automatically determined based on the target type, the direction of the modulation field, or both the target type and the direction of the modulation field.

In Example 3, the subject matter of Example 2 may optionally be configured such that the programming system is configured to present on a user interface for user selection more than one target type or more than one target type proxy corresponding to the more than one target type. The more than one target type may include at least one of an axon/dendrite cable, a cell, a terminal, or another pre-loaded target type, and present on a user interface a directional axis that can be moved via user interaction to define the direction of the modulation field. A proxy is a representation that serves as a substitution for the target type, such as an arrow or other representation. The user interaction may include dragging, toggling, moving the directional axis.

In Example 4, the subject matter of Example 3 may optionally be configured such that the programming system is configured to provide a target visualization corresponding to the user-selected target type or the user-selected target type proxy and impose a modulation field direction on the target visualization.

In Example 5, the subject matter of any one or more of Examples 1˜4 may optionally be configured such that the first user input identifies a location of the volume of neural tissue which corresponds to a target type geometry for the volume of neural tissue, and the stimulation configuration is automatically determined based on the identified location.

In Example 6, the subject matter of any one or more of Examples 1-5 may optionally be configured such that the volume of tissue includes at least one of a targeted volume or a side effect volume. The programming system may be configured to determine a spatial sensitivity for the at least one of the targeted volume or the side effect volume. The spatial sensitivity represents a proclivity of the volume of tissue to be affected by the delivered energy, and the stimulation configuration is determined using the determined spatial sensitivity.

In Example 7, the subject matter of Example 6 may optionally be configured such that the volume of tissue includes at least one of a targeted volume or a side effect volume. The programming system may be configured to determine a spatial sensitivity for the at least one of the targeted volume or the side effect volume. The spatial sensitivity represents a proclivity of the volume of tissue to be affected by the delivered energy, and the stimulation configuration is determined using the determined spatial sensitivity.

In Example 8, the subject matter of any one or more of Examples 1-7 may optionally be configured such that the programming system is further configured for receiving at least a second user input for spectrally controlling the broad-spectrum signal, spectrally adjusting the broad-spectrum signal based on the second user input to provide a spectrally-adjusted signal, and delivering energy corresponding to the spectrally-adjusted signal using the plurality of electrodes.

In Example 9, the subject matter of Example 8 may optionally be configured such that the energy is delivered using at least a first channel to deliver energy with a first phase to a first tissue volume and a second channel to deliver energy with a second phase to a second tissue volume, and wherein the first and second phases are different.

In Example 10, the subject matter of Example 9 may optionally be configured such that the second user input determines a phase for at least one of the first channel or the second channel.

In Example 11, the subject matter of any one or more of Examples 9-10 may optionally be configured such that the delivering energy further includes using at least a third channel to deliver energy with a third phase to a third tissue volume, where the first, second and third phases are different.

In Example 12, the subject matter of any one or more of Examples 9-11 may optionally be configured such that the first tissue volume includes a targeted volume of tissue and the second tissue volume includes a side effect volume of tissue. The first and second phases may be offset from each other by pi radians such that the energy delivered using the second channel has opposite polarity with respect to the energy delivered using the first channel.

In Example 13, the subject matter of any one or more of Examples 9-12 may optionally be configured such that the energy in the first channel is delivered through a first filter and the energy in the second channel is delivered through a second filter, and the first and second filters are configured to provide the different first and second phases.

In Example 14, the subject matter of Example 13 may optionally be configured such that the first and second filters are all-pass filters across the plurality of frequency ranges or have different frequency responses with different spectral band profiles across the plurality of frequency ranges.

In Example 15, the subject matter of any one or more of Examples 1-14 may optionally be configured such that the broad-spectrum signal is spectrally-adjusted with a spectral band profile corresponding to a type of tissue volume.

An example (e.g., Example 16) includes subject matter (such as a method, means for performing acts, machine readable medium including instructions that when performed by a machine cause the machine to perform acts (e.g., perform a method), or an apparatus to perform). The subject matter may include receiving at least a first user input for targeting energy to a volume of tissue, wherein the energy corresponds to a broad-spectrum signal having a plurality of frequency ranges, determining a stimulation configuration, including the modulation parameter set, based on the first user input, and delivering the energy corresponding to the broad-spectrum signal using the plurality of electrodes according to the determined stimulation configuration.

In Example 17, the subject matter of Example 16 may optionally be configured such that the at least the first user input defines a target type for the volume of tissue and a direction of a modulation field generated by the delivered energy, and the stimulation configuration is automatically determined based on the target type, the direction of the modulation field, or both the target type and the direction of the modulation field.

In Example 18, the subject matter of Example 17 may optionally be configured to further include presenting on a user interface for user selection more than one target type or more than one target type proxy corresponding to the more than one target type, wherein the more than one target type includes at least one of an axon/dendrite cable, a cell, a terminal, or another pre-loaded target type.

In Example 19, the subject matter of any one or more of Examples 17-18 may optionally be configured to further include presenting on a user interface a directional axis that can be moved via user interaction to define the direction of the modulation field.

In Example 20, the subject matter of any one or more of Examples 16-19 may optionally be configured to further include presenting on a user interface a target visualization corresponding to a user-selected target type or user-selected target type proxy and imposing a modulation field direction on the target visualization.

In Example 21, the subject matter of any one or more of Examples 16-20 may optionally be configured such that the at least the first user input identifies a location of the volume of neural tissue which corresponds to a target type geometry for the volume of neural tissue, and the stimulation configuration is automatically determined based on the identified location.

In Example 22, the subject matter of any one or more of Examples 16-21 may optionally be configured such that the volume of tissue includes at least one of a targeted volume or a side effect volume, the subject matter further includes determining a spatial sensitivity for the at least one of the targeted volume or the side effect volume, where the spatial sensitivity represents a proclivity of the volume of tissue to be affected by the delivered energy, and the stimulation configuration is determined using the determined spatial sensitivity.

In Example 23, the subject matter of Example 22 may optionally be configured such that the determining the spatial sensitivity includes determining one or more spatial sensitivities for one more targeted volumes and determining one or more spatial sensitivities for one or more side effect volumes.

Example 24, the subject matter of any one or more of Examples 16-23 may optionally be configured to further include receiving at least a second user input for spectrally controlling the broad-spectrum signal, spectrally adjusting the broad-spectrum signal based on the second user input to provide a spectrally-adjusted signal, and delivering energy corresponding to the spectrally-adjusted signal using a plurality of electrodes.

In Example 25, the subject matter of Example 24 may optionally be configured such that the delivering energy includes using at least a first channel to deliver energy with a first phase to a first tissue volume and a second channel to deliver energy with a second phase to a second tissue volume, and wherein the first and second phases are different.

In Example 26, the subject matter of Example 25 may optionally be configured such that the second user input determines a phase for at least one of the first channel or the second channel.

In Example 27, the subject matter of any one or more of Examples 25-26 may optionally be configured such that the delivering energy further includes using at least a third channel to deliver energy with a third phase to a third tissue volume. The first, second and third phases are different.

In Example 28, the subject matter of any one or more of Examples 25-27 may optionally be configured such that the first tissue volume includes a targeted volume of tissue and the second tissue volume includes a side effect volume of tissue. The first and second phases are offset from each other by pi radians such that the energy delivered using the second channel has opposite polarity with respect to the energy delivered using the first channel.

In Example 29, the subject matter of any one or more of Examples 25-28 may optionally be configured such that the energy in the first channel is delivered through a first filter and the energy in the second channel is delivered through a second filter. The first and second filters are configured to provide the different first and second phases.

In Example 30, the subject matter of Example 29 may optionally be configured such that the first and second filters are all-pass filters across the plurality of frequency ranges.

In Example 31, the subject matter of Example 29 may optionally be configured such that the first and second filters have different frequency responses with different spectral band profiles across the plurality of frequency ranges.

In Example 32, the subject matter of any one or more of Examples 24-31 may optionally be configured such that the broad-spectrum signal is spectrally-adjusted with a spectral band profile corresponding to a type of tissue volume.

In Example 33, the subject matter of any one or more of Examples 16-32 may optionally be configured such that the broad-spectrum signal includes a waveform pattern modulated by a modulation signal to provide a time varying-pattern, wherein the time varying pattern includes at least one of a dynamic pulse width, a dynamic amplitude or a dynamic frequency. The subject matter may further include receiving feedback, and adjusting at least one of the waveform pattern or the modulation signal based on the received feedback.

In Example 34, the subject matter of any one or more of Examples 16-33 may optionally be configured such that the broad-spectrum signal is a broad-spectrum digital signal. The subject matter may further include converting the broad-spectrum digital signal into a broad-spectrum analog signal, and introducing dithering noise into the broad-spectrum analog signal to provide a modified broad-spectrum analog signal. The delivered energy corresponds to the modified broad-spectrum analog signal using a plurality of electrodes.

An example (e.g., Example 35) includes subject matter (such as a method, means for performing acts, machine readable medium including instructions that when performed by a machine cause the machine to perform acts (e.g., perform a method), or an apparatus to perform). The subject matter may include receiving user input for spectrally controlling a broad-spectrum signal having a plurality of frequency ranges, spectrally adjusting the broad-spectrum signal based on the user input to provide a spectrally-adjusted signal, and delivering energy corresponding to the spectrally-adjusted signal using a plurality of electrodes.

In Example 36, the subject matter of Example 35 may optionally be configured such that the delivering energy includes using at least a first channel to deliver energy with a first phase to a first tissue volume and a second channel to deliver energy with a second phase to a second tissue volume. The first and second phases are different.

In Example 37, the subject matter of Example 36 may optionally be configured such that the user input determines a phase for at least one of the first channel or the second channel.

In Example 38, the subject matter of any one or more of Examples 36-37 may optionally be configured such that the delivering energy further includes using at least a third channel to deliver energy with a third phase to a third tissue volume. The first, second and third phases are different.

In Example 39, the subject matter of any one or more of Examples 35-38 may optionally be configured such that the first tissue volume includes a targeted volume of tissue and the second tissue volume includes a side effect volume of tissue. The first and second phases are offset from each other by pi radians.

In Example 40, the subject matter of any one or more of Examples 36-39 may optionally be configured such that the energy in the first channel is delivered through a first filter and the energy in the second channel is delivered through a second filter. The first and second filters are configured to provide the different first and second phases.

In Example 41, the subject matter of Example 40 may optionally be configured such that the first and second filters are all-pass filters across the plurality of frequency ranges.

In Example 42, the subject matter of Example 40 may optionally be configured such that the first and second filters have different frequency responses with different spectral band profiles across the plurality of frequency ranges.

In Example 43, the subject matter of any one or more of Examples 35-42 may optionally be configured such that the user input includes a type of tissue volume, and the broad-spectrum signal is spectrally-adjusted with a spectral band profile corresponding to the user-inputted type of tissue volume.

An example (e.g., Example 44) includes subject matter (such as a method, means for performing acts, machine readable medium including instructions that when performed by a machine cause the machine to perform acts (e.g., perform a method), or an apparatus to perform). The subject matter may include delivering energy corresponding to a broad-spectrum signal having a plurality of frequency ranges using a plurality of electrodes. The broad-spectrum signal includes a waveform pattern modulated by a modulation signal to provide a time varying-pattern. The subject matter may further include receiving feedback, and adjusting at least one of the waveform pattern or the modulation signal based on the received feedback.

In Example 45, the subject matter of Example 44 may optionally be configured such that the time varying pattern includes at least one of a dynamic pulse width, a dynamic amplitude or a dynamic frequency.

An example (e.g., Example 46) includes subject matter (such as a method, means for performing acts, machine readable medium including instructions that when performed by a machine cause the machine to perform acts (e.g., perform a method), or an apparatus to perform). The subject matter may include accessing a broad-spectrum digital signal having a plurality of frequency ranges, converting the broad-spectrum digital signal into a broad-spectrum analog signal, introducing dithering noise into the broad-spectrum analog signal to provide a modified broad-spectrum analog signal, and delivering energy corresponding to the modified broad-spectrum analog signal using a plurality of electrodes. This example may be combined with any other of the Examples.

An example (e.g., Example 47) Example includes subject matter (such as a method, means for performing acts, machine readable medium including instructions that when performed by a machine cause the machine to perform acts (e.g., perform a method), or an apparatus to perform). The subject matter may include sensing a broad-spectrum analog signal having a plurality of frequency ranges, introducing dithering noise in the broad-spectrum analog signal to provide a modified sensed signal, and converting the modified sensed signal into a digital signal. This example may be combined with any other of the Examples.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an embodiment of a neuromodulation system.

FIG. 2 illustrates an embodiment of a modulation device, such as may be implemented in the neuromodulation system of FIG. 1.

FIG. 3 illustrates an embodiment of a programming system such as a programming device, which may be implemented as the programming device in the neuromodulation system of FIG. 1.

FIG. 4 illustrates, by way of example, an embodiment of a SCS system, which also may be referred to as a Spinal Cord Modulation (SCM) system.

FIG. 5 illustrates, by way of example and not limitation, a flow diagram for spatially constraining a broad-spectrum signal.

FIG. 6 illustrates, by way of example and not limitation, a flow diagram for spectrally controlling a broad-spectrum signal.

FIG. 7 illustrates, by way of example and not limitation, a flow diagram for providing closed loop control for broad-spectrum signals.

FIG. 8 illustrates, by way of example and not limitation, a flow diagram for adding dithering noise to reduce quantization noise.

FIG. 9 illustrates, by way of example and not limitation, a flow diagram for adding dithering noise to a sensed broad-spectrum analog signal to reduce quantization noise.

FIG. 10 illustrates, by way of example and not limitation, a flow diagram for spatially and spectrally controlling a broad-spectrum signal with closed loop control and added dithering noise.

FIG. 11 illustrates, by way of example and not limitation, a programming screen for use to determine modulation parameters to be programmed into the modulation device.

FIG. 12 illustrates, by way of example and not limitation, a programming screen for broad-spectrum signals.

FIG. 13 illustrates, by way of example and not limitation, a user interface for defining a target type and a direction of the modulation field and visualizing a target field imposed on neural target.

FIG. 14 illustrates, by way of example and not limitation, determining spatial sensitivity (ies) for a neural target region(s) and/or side effect region(s) in relation to stimulation electrode(s).

FIG. 15 illustrates, by way of example and not limitation, the use of phases to selectively stimulation a neural target region(s) and avoid a side effect region(s).

FIG. 16 illustrates, by way of example and not limitation, a user interface for selecting or otherwise entering terminal biophysics and corresponding terminal sensitivity, and displaying a sensitivity frequency response to the selected biophysics.

FIG. 17 illustrates, by way of example and not limitation, a sensitivity of a target and a waveform filter that corresponds to the sensitivity of the target.

FIG. 18 illustrates, by way of example and not limitation, a sensitivity of a side effect and a waveform filter that inversely corresponds to the sensitivity of the side effect.

FIG. 19 illustrates, by way of example and not limitation, a decision tree for making spectral adjustments to a broad-spectrum signal for therapy enhancement, therapy block or inhibition, or side effect block or inhibition.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject matter includes systems and methods for applying and controlling broad-spectrum signals, whether noise or otherwise, to structures in the nervous system to modify function and/or to alleviate pathologies. Various embodiments target and spatially constrain the signal. By way of example and not limitation, the signal may be spatially constrained through anodic/cathodic fractionalizations and/or through the application of phase filters. Various embodiments spectrally control a variety of signals. Power band modulation may be potentially based on physiologically relevant oscillations. Various embodiments provide closed loop control for broad-spectrum signals. Various embodiments use dithering to reduce quantization noise. Although “noise” is used as the example, the concepts described here may be applicable to any signal type for which there is a broader spectrum or multiple frequency ranges.

The term “broad-spectrum” is intended to include signals with a plurality of frequency ranges, and may include frequency range(s) at very low, near-DC oscillations all the way up to blocking and tissue heating thresholds. The broad-spectrum signal may include noise or stochastic signals. Examples of types of noise signals include Gaussian, white, or 1/f noise where the energy level decreases as the frequency increases, such as by way of example pink or brown noise. White noise equally distributes the energy over all frequencies, whereas pink and brown noise does not equally distribute the energy over the frequencies. The energy for pink noise decreases with increasing frequency, and the energy for brown noise decreases faster with increasing frequency. By way of example, a high pass cutoff may be quite low, down to 0.1 Hz even though the 0 Hz DC component to be zero i.e., for the noise to be “zero mean,” as DC offset can lead to neuronal and, eventually, electrochemical consequences. A low pass cutoff(s) may use one or more of the following values. “High frequency block” starts to occur at or above 5-10 kHz. Above 30 kHz, substantial energy needs to be delivered to get activation or blocking responses. At frequencies above 100 kHz, the primary response is heating of the tissue, rather than neural activation.

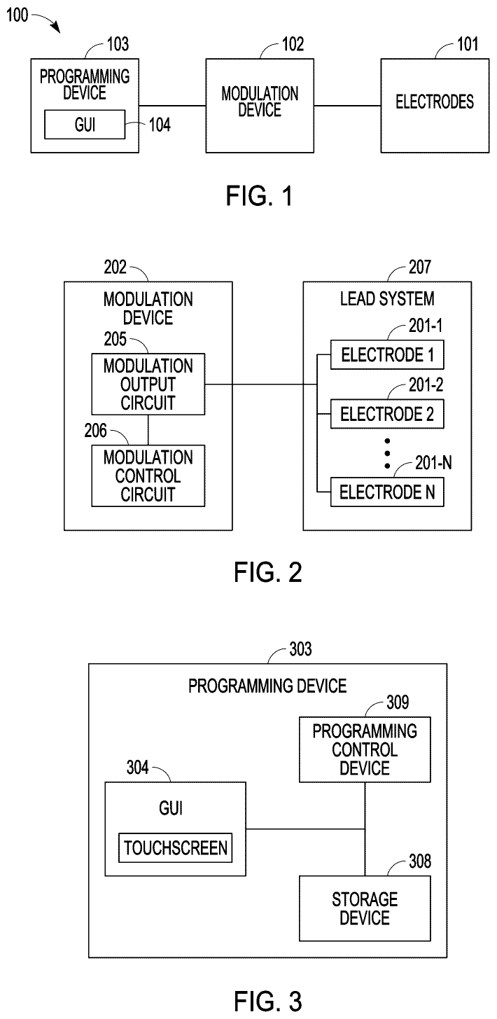

FIG. 1 illustrates, by way of example and not limitation, an embodiment of a neuromodulation system. The illustrated system 100 includes electrodes 101, a modulation device 102, and a programming system such as a programming device 103. The programming system may include multiple devices. The electrodes 101 are configured to be placed on or near one or more neural targets in a patient. The modulation device 102 is configured to be electrically connected to electrodes 101 and deliver neuromodulation energy, such as in the form of electrical pulses, to the one or more neural targets though electrodes 101. The delivery of the neuromodulation is controlled using a stimulation configuration. The stimulation configuration may include a modulation parameter set to specify the electrical waveform (e.g., pulses or pulse patterns or other waveform shapes). The stimulation configuration may also include an electrode configuration (e.g., selection of active electrodes through which the electrical waveform is delivered, the polarity of active electrodes, and the fractionalization to determine the energy distribution among the active electrodes. The modulation parameter set may also define the electrode configuration. The stimulation configuration may also include pulse timing information, such as a time delay and phase offset for a stimulation channel. In various embodiments, at least some parameters of the plurality of modulation parameters are programmable by a user, such as a physician or other caregiver. The programming device 103 provides the user with accessibility to the user-programmable parameters. In various embodiments, the programming device 103 is configured to be communicatively coupled to modulation device via a wired or wireless link. In various embodiments, the programming device 103 includes a graphical user interface (GUI) 104 that allows the user to set and/or adjust values of the user-programmable modulation parameters.

FIG. 2 illustrates an embodiment of a modulation device 202, such as may be implemented in the neuromodulation system 100 of FIG. 1. The illustrated embodiment of the modulation device 202 includes a modulation output circuit 205 and a modulation control circuit 206. Those of ordinary skill in the art will understand that the neuromodulation system may include additional components such as sensing circuitry for patient monitoring and/or feedback control of the therapy, telemetry circuitry and power. The modulation output circuit 205 produces and delivers the neuromodulation. Neuromodulation pulses are provided herein as an example. However, the present subject matter is not limited to pulses, but may include other electrical waveforms (e.g., waveforms with different waveform shapes, and waveforms with various pulse patterns). The modulation control circuit 206 controls the delivery of the neuromodulation pulses using the plurality of modulation parameters. The lead system 207 includes one or more leads each configured to be electrically connected to modulation device 202 and a plurality of electrodes 201-1 to 201-N distributed in an electrode arrangement using the one or more leads. Each lead may have an electrode array consisting of two or more electrodes, which also may be referred to as contacts. Multiple leads may provide multiple electrode arrays to provide the electrode arrangement. Each electrode is a single electrically conductive contact providing for an electrical interface between modulation output circuit 205 and tissue of the patient, where N≥2. The neuromodulation pulses are each delivered from the modulation output circuit 205 through a set of electrodes selected from the electrodes 201-1 to 201-N. The number of leads and the number of electrodes on each lead may depend on, for example, the distribution of target(s) of the neuromodulation and the need for controlling the distribution of electric field at each target. In one embodiment, by way of example and not limitation, the lead system includes two leads each having eight electrodes. Some embodiments may use a lead system that includes a paddle lead.

The actual number and shape of leads and electrodes may vary for the intended application. An implantable waveform generator may include an outer case for housing the electronic and other components. The outer case may be composed of an electrically conductive, biocompatible material, such as titanium, that forms a hermetically-sealed compartment wherein the internal electronics are protected from the body tissue and fluids. In some cases, the outer case may serve as an electrode (e.g., case electrode). The waveform generator may include electronic components, such as a controller/processor (e.g., a microcontroller), memory, a battery, telemetry circuitry, monitoring circuitry, modulation output circuitry, and other suitable components known to those skilled in the art. The microcontroller executes a suitable program stored in memory, for directing and controlling the neuromodulation performed by the waveform generator. A stimulation configuration determines how energy is delivered through the electrodes. A stimulation configuration may include a modulation parameter set which may include amplitude, pulse width, and frequency, an electrode configuration which may include fractionalization and polarity, and pulse timing which may include a time delay and phase offset. Electrical modulation energy is provided to the electrodes in accordance with a set of modulation parameters programmed into the pulse generator. By way of example but not limitation, the electrical modulation energy may be in the form of a pulsed electrical waveform. The stimulation configuration may comprise electrode combinations, which define the electrodes that are activated as anodes (positive), cathodes (negative), and turned off (zero), percentage of modulation energy assigned to each electrode (fractionalized electrode configurations), and electrical pulse parameters, which define the pulse amplitude (measured in milliamps or volts depending on whether the pulse generator supplies constant current or constant voltage to the electrode array), pulse width (measured in microseconds), pulse rate (measured in pulses per second), and burst rate (measured as the modulation on duration X and modulation off duration Y). Electrodes that are selected to transmit or receive electrical energy are referred to herein as “activated,” while electrodes that are not selected to transmit or receive electrical energy are referred to herein as “non-activated.”

Electrical modulation occurs between or among a plurality of activated electrodes, one of which may be the case of the waveform generator. The system may be capable of transmitting modulation energy to the tissue in a monopolar or multipolar (e.g., bipolar, tripolar, etc.) fashion. Monopolar modulation occurs when a selected one of the lead electrodes is activated along with the case of the waveform generator, so that modulation energy is transmitted between the selected electrode and case. Any of the electrodes E1-E16 and the case electrode may be assigned to up to k possible groups or timing “channels.” In one embodiment, k may equal four. The timing channel identifies which electrodes are selected to synchronously source or sink current to create an electric field in the tissue to be stimulated. Amplitudes and polarities of electrodes on a channel may vary. In particular, the electrodes can be selected to be positive (anode, sourcing current), negative (cathode, sinking current), or off (no current) polarity in any of the k timing channels. The waveform generator may be operated in a mode to deliver electrical modulation energy that is therapeutically effective and causes the patient to perceive delivery of the energy (e.g., therapeutically effective to relieve pain with perceived paresthesia), and may be operated in a sub-perception mode to deliver electrical modulation energy that is therapeutically effective and does not cause the patient to perceive delivery of the energy (e.g., therapeutically effective to relieve pain without perceived paresthesia).

The waveform generator may be configured to individually control the magnitude of electrical current flowing through each of the electrodes. For example, a current generator may be configured to selectively generate individual current-regulated amplitudes from independent current sources for each electrode. In some embodiments, the pulse generator may have voltage regulated outputs. While individually programmable electrode amplitudes are desirable to achieve fine control, a single output source switched across electrodes may also be used, although with less fine control in programming. Neuromodulators may be designed with mixed current and voltage regulated devices.

The neuromodulation system may be configured to modulate spinal target tissue or other neural tissue. The configuration of electrodes used to deliver electrical pulses to the targeted tissue constitutes an electrode configuration, with the electrodes capable of being selectively programmed to act as anodes (positive), cathodes (negative), or left off (zero). In other words, an electrode configuration represents the polarity being positive, negative, or zero. An electrical waveform may be controlled or varied for delivery using electrode configuration(s). The electrical waveforms may be analog or digital signals. In some embodiments, the electrical waveform includes pulses. The pulses may be delivered in a regular, repeating pattern, or may be delivered using complex patterns of pulses that appear to be irregular. Other parameters that may be controlled or varied include the amplitude, pulse width, and rate (or frequency) of the electrical pulses. Each electrode configuration, along with the electrical pulse parameters, can be referred to as a “modulation parameter set.” Each set of modulation parameters, including fractionalized current distribution to the electrodes (as percentage cathodic current, percentage anodic current, or off), may be stored and combined into a modulation program that can then be used to modulate multiple regions within the patient.

The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. Furthermore, for example SCS systems may have thirty-two electrodes which exponentially increases the number of modulation parameters sets available for programming. To facilitate such selection, the clinician generally programs the modulation parameters sets through a computerized programming system to allow the optimum modulation parameters to be determined based on patient feedback or other means and to subsequently program the desired modulation parameter sets.

FIG. 3 illustrates an embodiment of a programming system such as a programming device 303, which may be implemented as the programming device 103 in the neuromodulation system of FIG. 1. The programming device 303 includes a storage device 308, a programming control circuit 309, and a graphical user interface (GUI) 304. The programming control circuit 309 generates the plurality of modulation parameters that control the delivery of the neuromodulation pulses according to the pattern of the neuromodulation pulses. In various embodiments, the GUI 304 includes any type of presentation device, such as interactive or non-interactive screens, and any type of user input devices that allow the user to program the modulation parameters, such as touchscreen, keyboard, keypad, touchpad, trackball, joystick, and mouse. The storage device 308 may store, among other things, modulation parameters to be programmed into the modulation device. The programming device 303 may transmit the plurality of modulation parameters to the modulation device. In some embodiments, the programming device 303 may transmit power to the modulation device. The programming control circuit 309 may generate the plurality of modulation parameters. In various embodiments, the programming control circuit 309 may check values of the plurality of modulation parameters against safety rules to limit these values within constraints of the safety rules.

In various embodiments, circuits of neuromodulation, including its various embodiments discussed in this document, may be implemented using a combination of hardware, software and firmware. For example, the circuit of GUI, modulation control circuit, and programming control circuit, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

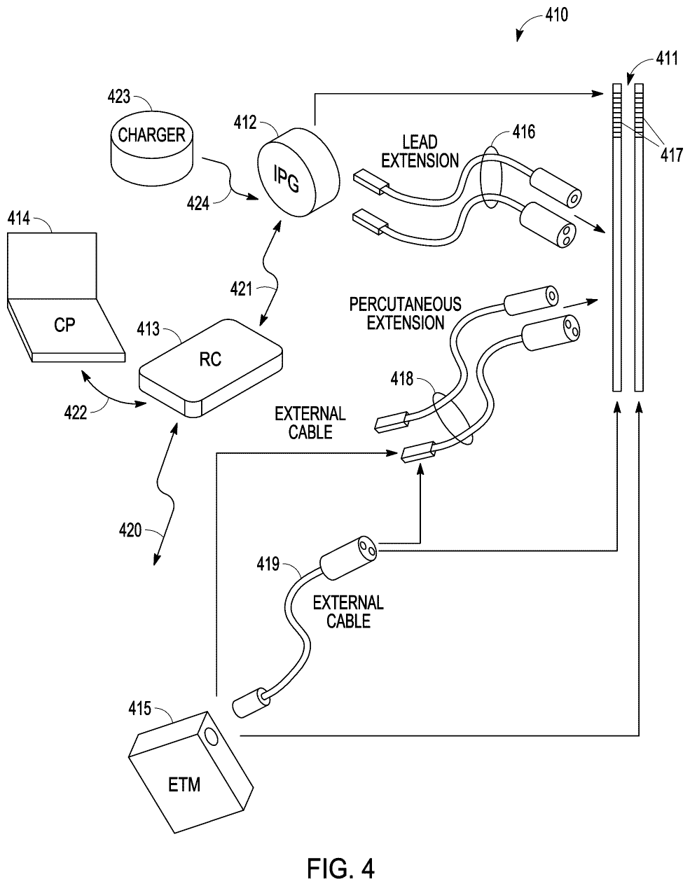

FIG. 4 illustrates, by way of example, an embodiment of a SCS system, which also may be referred to as a Spinal Cord Modulation (SCM) system. The SCS system 410 may generally include one or more (illustrated as two) of implantable neuromodulation leads 411, an electrical waveform generator 412, an external remote controller (RC) 413, a clinician's programmer (CP) 414, and an external trial modulator (ETM) 415. IPGs are used herein as an example of the electrical waveform generator. However, it is expressly noted that the waveform generator may be configured to deliver regular, repeating patterns of pulses or in complex patterns that appear to be irregular patterns of pulses where pulses have differing amplitudes, pulse widths, pulse intervals, and bursts with differing number of pulses. It is also expressly noted that the waveform generator may be configured to deliver electrical waveforms other than pulses. The waveform generator 412 may be physically connected via one or more percutaneous lead extensions 416 to the neuromodulation leads 411, which carry a plurality of electrodes 417. As illustrated, the neuromodulation leads 411 may be percutaneous leads with the electrodes arranged in-line along the neuromodulation leads. Any suitable number of neuromodulation leads can be provided, including only one, as long as the number of electrodes is greater than two (including the waveform generator case function as a case electrode) to allow for lateral steering of the current. Alternatively, a surgical paddle lead can be used in place of one or more of the percutaneous leads. In some embodiments, the waveform generator 412 may include pulse generation circuitry that delivers electrical modulation energy in the form of a pulsed electrical waveform (i.e., a temporal series of electrical pulses) to the electrodes in accordance with a set of modulation parameters.

The ETM 415 may also be physically connected via the percutaneous lead extensions 418 and external cable 419 to the neuromodulation leads 411. The ETM 415 may have similar waveform generation circuitry as the waveform generator 412 to deliver electrical modulation energy to the electrodes in accordance with a set of modulation parameters. The ETM 415 is a non-implantable device that is used on a trial basis after the neuromodulation leads 411 have been implanted and prior to implantation of the waveform generator 412, to test the responsiveness of the modulation that is to be provided. Functions described herein with respect to the waveform generator 412 can likewise be performed with respect to the ETM 415.

The RC 413 may be used to telemetrically control the ETM 415 via a bi-directional RF communications link 420. The RC 413 may be used to telemetrically control the waveform generator 412 via a bi-directional RF communications link 421. Such control allows the waveform generator 412 to be turned on or off and to be programmed with different modulation parameter sets. The waveform generator 412 may also be operated to modify the programmed modulation parameters to actively control the characteristics of the electrical modulation energy output by the waveform generator 412. A clinician may use the CP 414 to program modulation parameters into the waveform generator 412 and ETM 415 in the operating room and in follow-up sessions. The waveform generator 412 may be implantable. The implantable waveform generator 412 and the ETM 415 may have similar features as discussed with respect to the modulation device 202 described with respect to FIG. 2.

The CP 414 may indirectly communicate with the waveform generator 412 or ETM 415, through the RC 413, via an IR communications link 422 or other link. The CP 414 may directly communicate with the waveform generator 412 or ETM 415 via an RF communications link or other link (not shown). The clinician detailed modulation parameters provided by the CP 414 may also be used to program the RC 413, so that the modulation parameters can be subsequently modified by operation of the RC 413 in a stand-alone mode (i.e., without the assistance of the CP 414). Various devices may function as the CP 414. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 414. Alternatively, such programming methodologies can be performed using firmware or hardware. In any event, the CP 414 may actively control the characteristics of the electrical modulation generated by the waveform generator 412 to allow the desired parameters to be determined based on patient feedback or other feedback and for subsequently programming the waveform generator 412 with the desired modulation parameters. To allow the user to perform these functions, the CP 518 may include a user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant waveform generator, implant waveform generator and lead(s), replace waveform generator, replace waveform generator and leads, replace or revise leads, explant, etc.), generate a pain map of the patient, define the configuration and orientation of the leads, initiate and control the electrical modulation energy output by the neuromodulation leads, and select and program the IPG with modulation parameters in both a surgical setting and a clinical setting.

An external charger 423 may be a portable device used to transcutaneously charge the waveform generator via a wireless link such as an inductive link 424. Once the waveform generator has been programmed, and its power source has been charged by the external charger or otherwise replenished, the waveform generator may function as programmed without the RC or CP being present.

The present subject matter includes systems and methods for applying and controlling broad-spectrum signals, whether noise, random, or stochastic signals or signals corresponding to stimulation patterns modulated by function(s)). The broad-spectrum signals may be applied to structures in the nervous system to modify function and/or to alleviate pathologies. The term “broad-spectrum” is intended to include signals with a plurality of frequency ranges, and may include frequency range(s) near a lower range near very low, near-DC oscillations, may include frequency range(s) near a higher range near blocking and tissue heating thresholds, and may include smaller subsets of frequency ranges between the lower and upper ranges. By way example and not limitation, a range of frequencies may include any range within a range from near DC (e.g., 0.1 Hz) to 100 kHz). Non-limiting examples of smaller subsets of frequency ranges may include a range extending from 0.1 Hz to an upper range above 1.5 to 5 kHz, or a range from 10 Hz to about 1,200 Hz. The range of frequencies for the broad-spectrum signal need not extend all the way to near DC (e.g., 0.1 Hz) and need not extend all the way to the upper limit (e.g., 5 kHz, 10 kHz, 30 kHz, 100 kHz, etc.).

Various embodiments target and spatially constrain the signal. A basis for spatially constraining the signal is that neurological or other targets may vary in their sensitivity to electric fields according to their individual geometries. By way of example and not limitation, the signal may be spatially constrained through anodic/cathodic fractionalizations and/or through the application of phase filters.

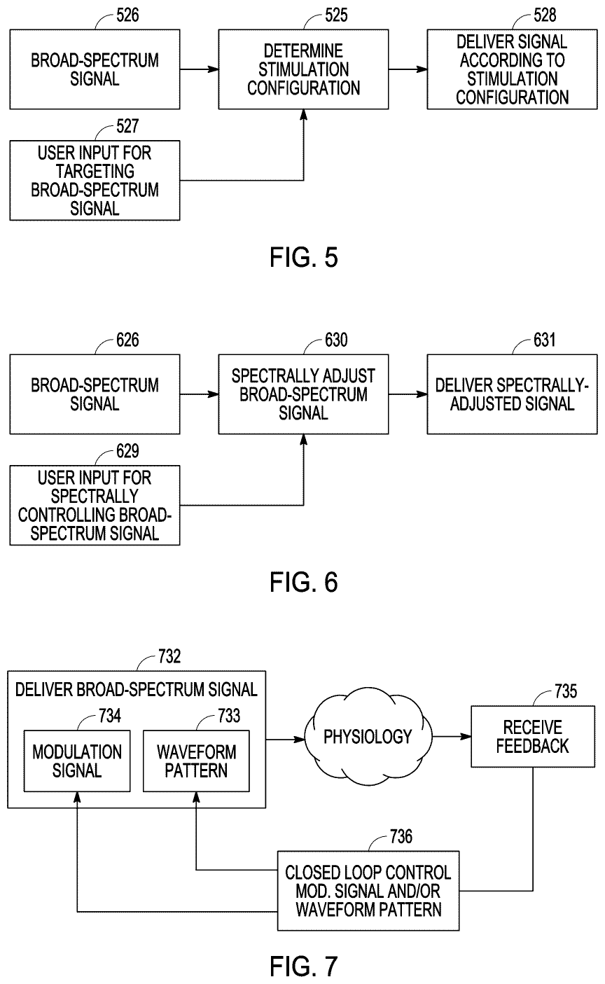

FIG. 5 illustrates, by way of example and not limitation, a flow diagram for spatially constraining a broad-spectrum signal. A stimulation configuration is determined at 525 for a broad-spectrum signal 526 based on received user input 527 for targeting the broad-spectrum signal. At 528, a signal is delivered according to the determined stimulation configuration. A method performed by the present subject matter may include receiving at least a first user input 527 for targeting energy to a volume of tissue. The energy corresponds to a broad-spectrum signal 526 having a plurality of frequency ranges. The method may include determining a stimulation configuration 525 (e.g., the modulation parameter set or electrode configuration such as electrode selection, polarity, fractionalization), based on the first user input, and delivering the energy 528 corresponding to the broad-spectrum signal using the plurality of electrodes according to the determined stimulation configuration. A system may include a plurality of electrodes, a neural modulation device configured to deliver energy, according to a modulation parameter set, using at least some of the plurality of electrodes to modulate a volume of neural tissue.

FIG. 6 illustrates, by way of example and not limitation, a flow diagram for spectrally controlling a broad-spectrum signal. Power band modulation may be potentially based on physiologically relevant oscillations. A method may include receiving user input 629 for spectrally controlling a broad-spectrum signal 626 having a plurality of frequency ranges. The method may further include spectrally adjusting 630 the broad-spectrum signal based on the user input 629 to provide a spectrally-adjusted signal, and delivering energy 631 corresponding to the spectrally-adjusted signal using a plurality of electrodes.

FIG. 7 illustrates, by way of example and not limitation, a flow diagram for providing closed loop control for broad-spectrum signals. A method may include delivering energy corresponding to a broad-spectrum signal 732 having a plurality of frequency ranges using a plurality of electrodes. The broad-spectrum signal may include a waveform pattern 733 modulated by a modulation signal 734 to provide a time varying-pattern. The method may include receiving feedback 735 (e.g., via physiologic sensor(s)), and adjusting (via closed-loop control 736) at least one of the waveform pattern 733 or the modulation signal 734 based on the received feedback 735. By way of example and not limitation, the waveform pattern may include pulses with an amplitude, pulse width, pulse frequency, burst duration and burst interval. Any one or more of these may be modulated using the modulation signal 734. The time varying pattern may include at least one of a dynamic pulse width, a dynamic amplitude or a dynamic frequency. It is noted that user input, such as answers to questions like “How do you feel?”, “Rate your pain?” by way of example and not limitation.

Parameter modulation with added noise selection use in a closed-loop control configuration. The system may use a manual or automatic closed loop. With manual, the patient selects the type of closed loop that provides the best outcome. With automatic, the algorithm automatically decides optimal closed loop approach. Time varying patterns may be used where the pattern is modulating any stimulation parameter such as amplitude, pulse width, frequency, duty cycle, charge per second, charge per phase, cycling, or others.

The broad-spectrum neuromodulation signal may be random noise that can operate in open loop, but can also be adjusted in a closed loop approach based on physiological sensing from implantable or external wearable sensors. Examples of such sensors include, but are not limited to, an accelerometer (+gyroscope), wrist activity measurement, skin impedance, pupil dilation, ECG, heart rate, heart rate variability, sleep/resting time, body temperature, oxygen level, glucose level, other chemical levels, electrodermal activity, eye movement (from framed glasses, sunglasses detectors, or other), eye movement during sleep sensor, etc. Based on the physiological measurement from the external or internal sensor the noise modulating signal may be adjusted to increase patient benefits such as reduction of pain, or increase of a comfortable stimulating sensation (massage, placid sensation) as opposed to tingling paresthesia sensation. The noise signal that is also constantly changing modifies the stimulation parameter(s) (e.g., at least one of frequency, amplitude, pulse width for pulses in a pulse pattern) that varies according to the noise changes. At the same time the noise changes according to the sensed information or according to a tuning approach. Examples of stimulation patterns modulated by a noise signal controlled by sensed information may include dynamic pulse width, dynamic amplitude and dynamic frequency. Although “noise” is used as the example, the concepts described here may be applicable to any signal type for which there is a broader spectrum or multiple frequency ranges.

Various embodiments use dithering to reduce quantization noise. One of the challenges when utilizing ADC (analog to digital converter) for sensing physiological or external signals via wearables or implantable sensors is the quantization error that takes place in this conversion. Similarly, when using a DAC (digital to analog converter) the analog signal created can show and have presence of discretization noise.

A technique highly used in other areas (e.g., audio, images) is randomizing the quantization error by adding dithering noise which can prevent large scale patterns such as signal spectral bands appearing in a time varying stimulation current delivered in spinal cord stimulators or brain stimulators, or peripheral nerves stimulators. This can make the DAC signal smoother closer to real biological responses, rather than having very precise and specific quantization jumps, it will be dither noise to soften the discretization error. The same approach applies when sensing, the ADC could undergo a post stage where dithering is added to smooth/reduce the effects of the quantization error.

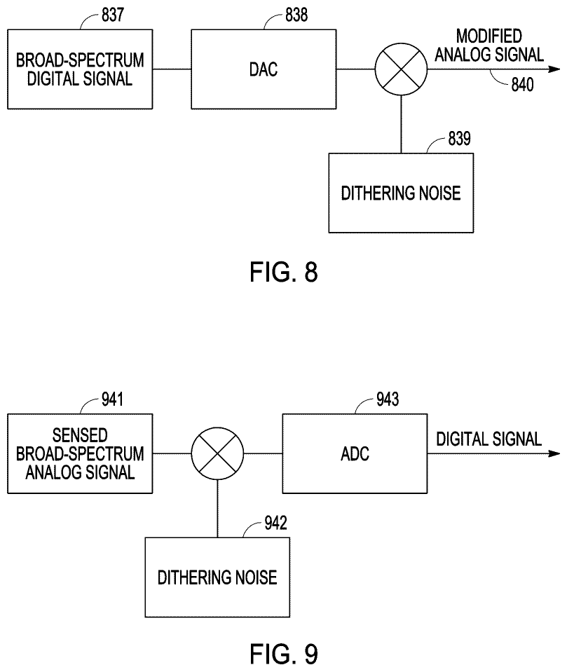

FIG. 8 illustrates, by way of example and not limitation, a flow diagram for adding dithering noise to reduce quantization noise. In the illustration, a method to reduce quantization noise in an signal corresponding to delivered neuromodulation energy may include accessing a broad-spectrum digital signal having a plurality of frequency ranges 837, converting the broad-spectrum digital signal into a broad-spectrum analog signal via a digital to analog converter (DAC) 838, introducing dithering noise 839 (e.g., an analog signal) into the broad-spectrum analog signal to provide a modified broad-spectrum analog signal, and delivering energy 840 corresponding to the modified broad-spectrum analog signal using a plurality of electrodes. It is noted that the dithering noise may be added as a digital signal to the broad-spectrum digital signal before the digital signal is converted to an analog signal.

FIG. 9 illustrates, by way of example and not limitation, a flow diagram for adding dithering noise to a sensed broad-spectrum analog signal to reduce quantization noise. In the illustration, a method to reduce quantization noise in a sensed signal may include sensing a broad-spectrum analog signal having a plurality of frequency ranges 941, introducing dithering noise 942 (e.g., analog signal) in the broad-spectrum analog signal to provide a modified sensed signal, and converting the modified sensed signal into a digital signal via an analog to digital converter (ADC) 943. It is noted that an example of a sensed broad-spectrum analog signal may be sensed nerve activity. It is also noted that the digital dithering signal may be added after the sensed broad-spectrum analog signal is converted to a digital signal.

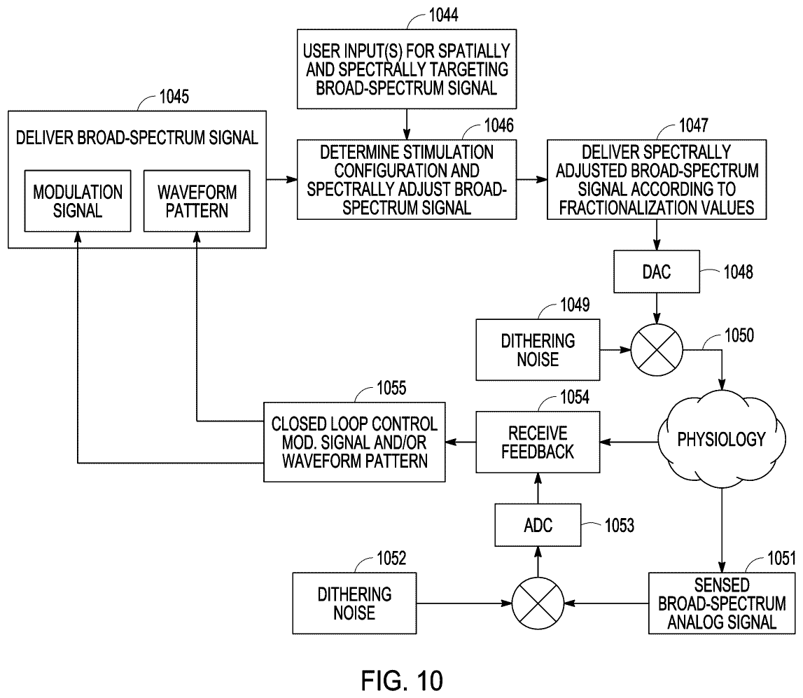

Any of the concepts discussed with respect to FIGS. 5-9 may be combined with one or more of the other concepts discussed with respect to these figures. FIG. 10 illustrates, by way of example and not limitation, a flow diagram for spatially and spectrally controlling a broad-spectrum signal with closed loop control and added dithering noise. A method performed by the present subject matter may include receiving at least a first user input 1044 for spatially and spectrally targeting energy to a volume of tissue, wherein the energy corresponds to a broad-spectrum signal 1045 having a plurality of frequency ranges, determining a stimulation configuration 1046 based on the first user input to provide a spatially targeted and spectrally adjusted broad-spectrum signal, which may be delivered 1047 according to the determined stimulation configuration. The spectrally adjusted broad-spectrum signal may be digital signal, which is converted into an analog signal via a DAC 1048. Dithering noise 1049 may be added to the neuromodulation signal 1050 that is applied to tissue.

Sensor(s) may be used to sense a broad-spectrum analog signal 1051. Dithering noise 1052 may be added to the sensed analog signal, and then converted from an analog signal to a digital signal via an ADC 1053. The digital signal may be received as feedback 1054 into a closed-loop control 1055 for modulating the broad-spectrum signal. For example, the feedback may be used to control modulation signal and/or the waveform pattern in the broad-spectrum signal.

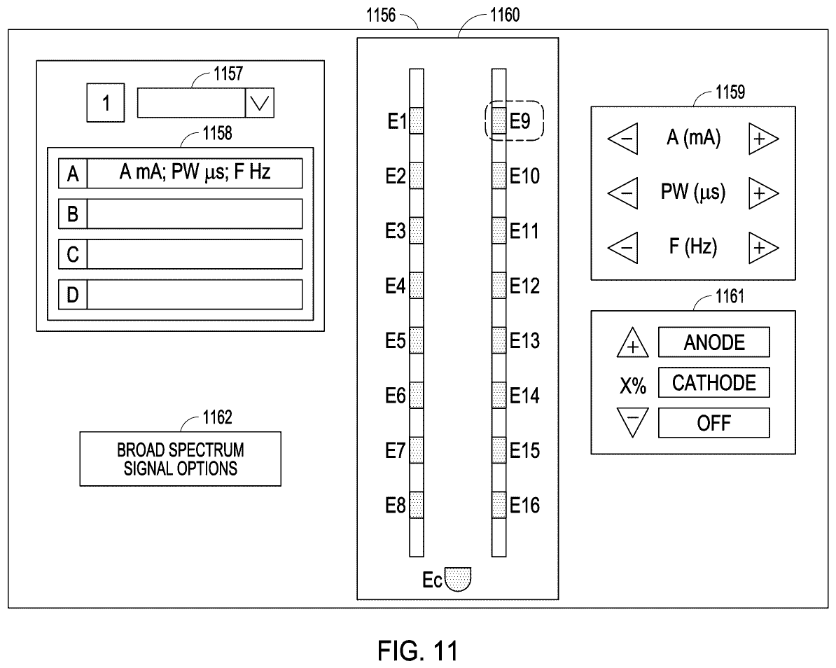

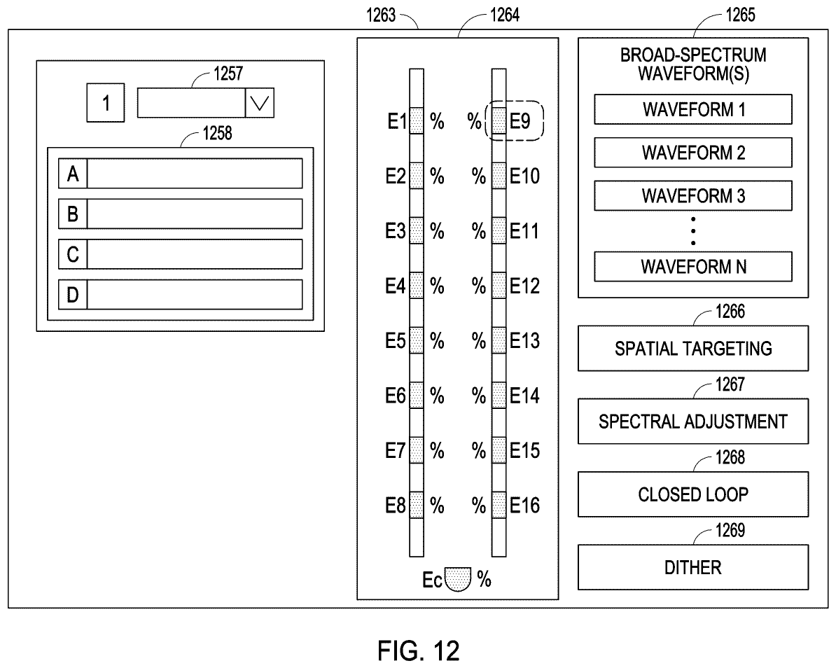

FIG. 11 illustrates, by way of example and not limitation, a programming screen for use to determine modulation parameters to be programmed into the modulation device. The system may be capable of storing and implementing different programs based on user input, feedback or a schedule. The illustrated programming screen 1156 includes a program selection 1157 element for use in selecting and/or programming a neuromodulation program. Each program may include one or more timing channels, which may also be referred to as stimulation channels 1158. There are four timing channels in the illustrated embodiment. Each timing channel identifies which electrodes are selected to synchronously source or sink current to create an electric field in the tissue to be stimulated, and also illustrates the waveform (pulse amplitude, pulse width, pulse frequency) for that channel's activated electrodes. These stimulation parameters for a selected channel may be programmed in region 1159. The center region 1160 allows the user to select the electrodes for the selected timing channel. Any of the electrodes E1-E16 or can electrode Ec may be selected, and the user may control whether the electrode is OFF (inactive), an anode, or a cathode via region 1161. Additionally, the user is able to determine or adjust the fractionalization for that electrode. The total anodic energy is 100% and the total cathodic energy is 100%. The system can distribute that total anodic energy over one or more active anodic electrodes and can distribute that total cathodic energy over one or more active cathodic electrodes. It is noted that, in some embodiments, stimulation configuration including the electrode selection and fractionalization may be preprogrammed and loaded based on a user input, such as the user inputs references for FIGS. 5, 6 and 10. The display on the user interface may include a button 1162 or other user interface element to allow a user to access broad-spectrum signal options. By way of example and not limitation, actuation of the button may change the display to a display similar to that which is illustrated in FIG. 12.

FIG. 12 illustrates, by way of example and not limitation, a programming screen for broad-spectrum signals. Similar to FIG. 11, the display 1263 of FIG. 12 may include a program selection 1257 and timing channels 1258 for each program. Also similar to FIG. 11, the display of FIG. 12 may include a region 1264 to provide an electrode configuration (e.g., fractionalization) for the selected timing channel. The user interface may include available broad-spectrum waveform(s) 1265 that are available for selection. These waveforms may include different modulated waveform patterns where one or more parameters of the waveform are modulated. The modulated waveform parameters may include waveform patterns with stochastically modulated parameter(s), waveform patterns with noise introduced in the waveform, waveform patterns modulated using one or more deterministic functions.

The programming screed for broad-spectrum signals may include buttons or other user interface elements for spatial targeting 1266 of the broad-spectrum waveform(s), spectral adjustment 1267 of the broad-spectrum waveform(s), closed-loop control 1268, and dithering 1269. Any of these may be actuated to allow the user to further constrain the selected broad-spectrum signal to be used to deliver neuromodulation.

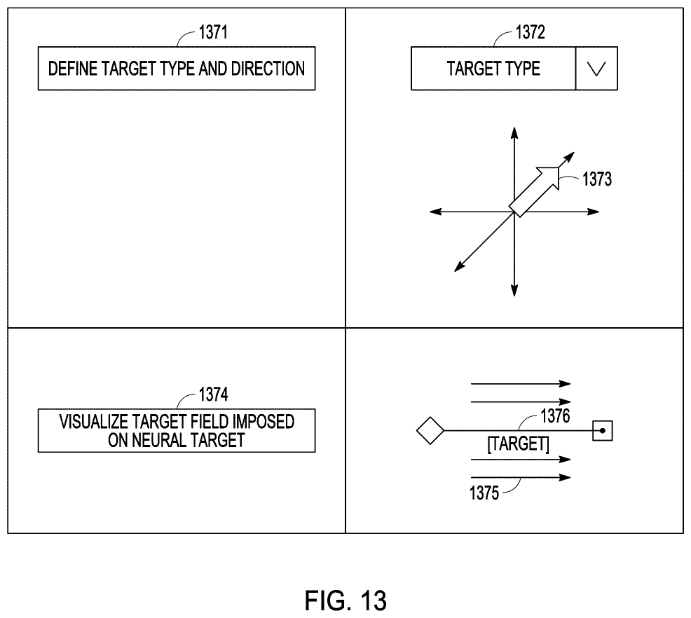

FIGS. 13-15 illustrate, by way of example and not limitation, various spatial targeting embodiments. A basis is that neurological or other targets vary in their sensitivity to electric fields according to their individual geometries. Fractionalizations/anode cathode splits and separations may be set according to the target via existing algorithms and/or first principles in neural engineering. Broad-spectrum (e.g., noise) signals may be applied through or with different fractionalizations. Broad-spectrum signals may be designed to constructively interfere with primary signal, to destructively interfere with it, and/or to create “composite” signals via fractionalization control. By way of example, to modulate axonal or dendritic terminals, the system may align an electric (i.e., first order voltage difference or uniform electric field) along the direction of propagation. To modulate axons or dendrite cables, the system may use anode or cathode configurations to “focus” stimulation or maximize second order voltage difference at specific nodes. Axons may also be blocked through a similar mechanism, accompanied by particular filter adjustments. Axon selectivity may be better modulated using “lateral” electrodes such as electrodes on a cuff. To modulate cells, the system may apply anodic stimulation to maximize (asymmetric) second difference at axon hillock of the element. However, cellular stimulation geometry depends on location (e.g., spinal vs. brain vs. peripheral ganglion). In the case of brain, the applied stimulation geometry may be affected by epicortical vs. deep brain. Geometric adjustments may need to be made to account for the “Total Equivalent Driving Function” (e.g., Warman E N, Grill W M, Durand D. Modeling the effects of electric fields on nerve fibers: determination of excitation thresholds. IEEE Trans Biomed Eng. 1992 December: 39 (12): 1244-54. doi: 10.1109/10.184700. PMID: 1487287.) for frequency components under 1 kHz (akin to pulse widths >1 ms), whereas quasi static “Activating function” can be used for frequency components above 1 kHz. Lower frequency components may “bleed between” Nodes of Ranvier along an axon or terminal. Geometries may be automatically configured according to region (brain, spinal cord, or peripheral nerve) and/or electrode attachment type. Modulation targets in the brain include axons, cells and terminals. Spinal cord modulation may emphasize modulation of axons, although some cell modulation is possible with “sub perception” modalities. Peripheral nerve modulation may only enable axon selectivity, but may differentiate between any axon diameter settings. Some interface elements are shown below

FIG. 13 illustrates, by way of example and not limitation, a user interface 1370 for defining a target type and a direction of the modulation field and visualizing a target field imposed on neural target. The target type and direction may be defined. For example, a define button 1371 or other user interface element may be actuated to open a window where a user can select a target type 1372 for the targeted tissue. FIG. 13 illustrates a pulldown selection, but other manners of selection may be used. Examples of selectable target types may include at least some of the following types: an axon cable, a dendrite cable, a cell, a terminal end, or a dendrite end. Other pre-stored target types may be used. Some embodiments may create the target types from tissue models. In some embodiments, a directional axis 1373 may be presented on a coordinate system. A user can drag, toggle, or otherwise move or manipulate the orientation of the direction axis with respect to the coordinate system. The illustrated coordinate system is a three-dimensional Cartesian coordinate system (X, Y. Z). The coordinate system may be identified using medial-lateral (ML) directions, dorsal-ventral (DV) directions, and rostral-caudal (RC) directions. Other coordinates may be used.

The system may be configured to display the target on the user interface for visualization. For example, a visualize target button 1374 or another user interface element may be actuated to display the target. The desired electric field 1375 be displayed over the displayed target 1376. The geometry of the target displayed on the user interface may be determined by the location (e.g., brain, spinal cord, peripheral) of the target.

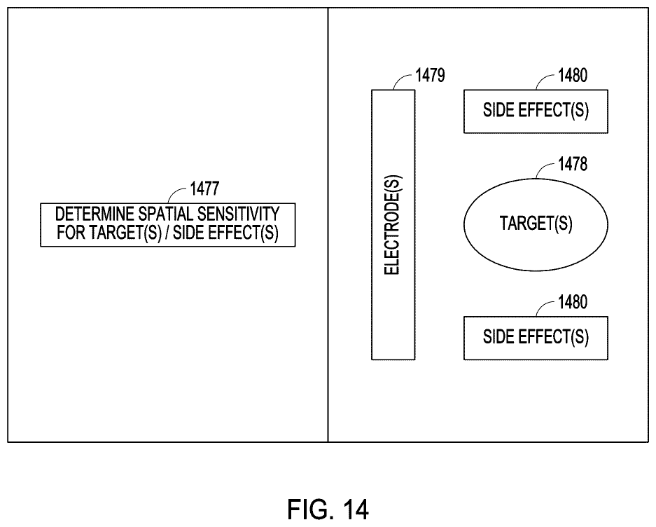

FIG. 14 illustrates, by way of example and not limitation, determining spatial sensitivity (ies) for a neural target region(s) and/or side effect region(s) in relation to stimulation electrode(s). For example, a determine spatial sensitivity button 1477 or other user interface element may be actuated to reconcile the target 1478 with the stimulating electrode(s) 1479. For near side effect region(s) 1480, anodes may be used to cancel fields/charges created by a central cathodic stimulation point at a target 1480 via destructive interference. Between the target region(s) 1478 and the side effect region(s) 1480, the signal may be tapered over space by grading the fractionalization and/or passing the signal through all-pass, phase delay filters. Thus, the change in the modulation field gradually changes between the targeted region(s) and the side effect region(s).

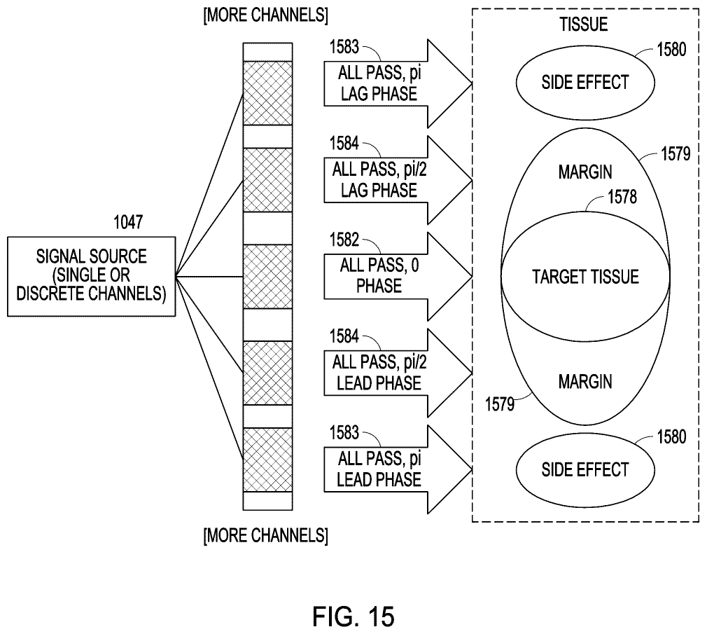

FIG. 15 illustrates, by way of example and not limitation, the use of phases to selectively stimulation a neural target region(s) and avoid a side effect region(s). The figure illustrates tissue with side effect regions 1580, target tissue region 1578 and margins 1581 between the target tissue region and the side effect regions. The modulation signal may be from a single source but channel filters may be used. The target tissue may be modulated by passing the modulation signal through an all-pass, 0 phase delay filter (or no filter) 1582 such that the modulation signal is applied to the target tissues. The side effect tissue may be modulated by passing the modulation signal through an all-pass, pi (180 deg.) lag filter or an all-pass, pi (180 deg.) lead filter 1583. The margin tissue regions may be modulated by passing the modulation signal through an all-pass pi/2 (90 deg.) lag filter or an all-pass pi/2 (90 deg.) lead filter 1584. The pi lag or pi lead offset is equivalent of setting an opposite sign for the fractionalization. The lead/lag interval (in radians, or in plain language like “offset” and “opposite”) may be configured by user. The interval need not be a fixed or constant value, nor does it have to be lead or lag as shown. The number of channels and/or physical length (translated or rounded into contact count or interval size) over which full lead/lag range occurs can be specified by user. The filter may not be all pass. The filter(s) may also include gain/loss elements to accommodate the sensitivity of the neural element. The filter(s) may be distinct depending on therapy/side effect generating elements. The filter(s) may also be designed with intentional phase distortion such that only some/specific frequency bands have lead/lag applied to them.

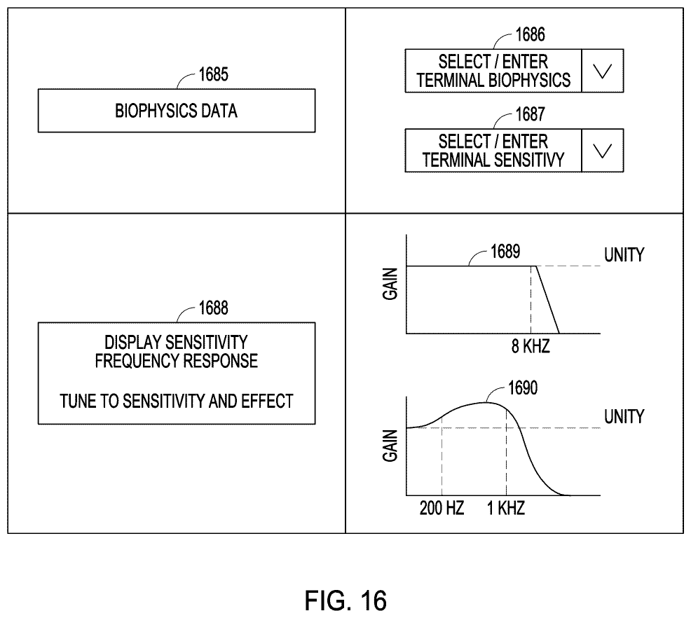

FIGS. 16-19 illustrate, by way of example and not limitation, various spectral adjustment embodiments. The frequency bands may be constrained or tuned based on the membrane sensitivity of the intended target. Target sensitivity, along with being geometry dependent, may also be depend on biophysics. The frequency band(s) over which noise or another signal may be applied may be automatically tuned according to the biophysics of the target in question. The broad-spectrum signal, such as but not limited to noise, may also be selected/attenuated based on biological range bands such as neural oscillations or brainwaves which include theta, alpha, beta, and low and high gamma. Signal properties can be determined through a decision process and/or user definition.

FIG. 16 illustrates, by way of example and not limitation, a user interface for selecting or otherwise entering terminal biophysics and corresponding terminal sensitivity, and displaying a sensitivity frequency response to the selected biophysics. For example, an option may be given to upload biophysics or otherwise to generate definition of membrane dynamics. Membrane dynamics, in turn, governs sensitivity to particular frequency bands. By way of example, a biophysics button 1685 or other user interface element may be actuated to open a window where a user can select or enter neural (e.g., terminal) biophysics 1686 and/or select or enter neural (e.g., terminal) sensitivity 1687. For example, a highly experienced or power user may enter terminal biophysics such as passive, Hodgkin-Huxley model, a McIntyre, Richardson and Grill (MRG) model, or other biophysics from a cloud. A lesser experience or casual user may enter terminal sensitivity such as inactive, low, medium, high, and or auto-recommended entry.

The system may be configured to display options for sensitivity. By way of example, a biophysics button 1688 or other user interface element may be actuated to open a window where a user can tune for the desired frequency effect. The illustrated figure includes Low/Passive sensitivity 1689 (by way of example and not limitation, unity until 8 kHz) and high sensitivity 1690 (e.g., above unity between 700 Hz and 1 kHz, with a block at higher frequencies. FI and F2 can typically range from 200 Hz to 8 kHz, as typical nerve membrane time constants are 1 ms 200 Hz corresponds to a period that 4× the time constant (i.e., full charge or drain) and the membrane may not charge substantially when the frequency is greater than approximately 8 to 10 KHz.

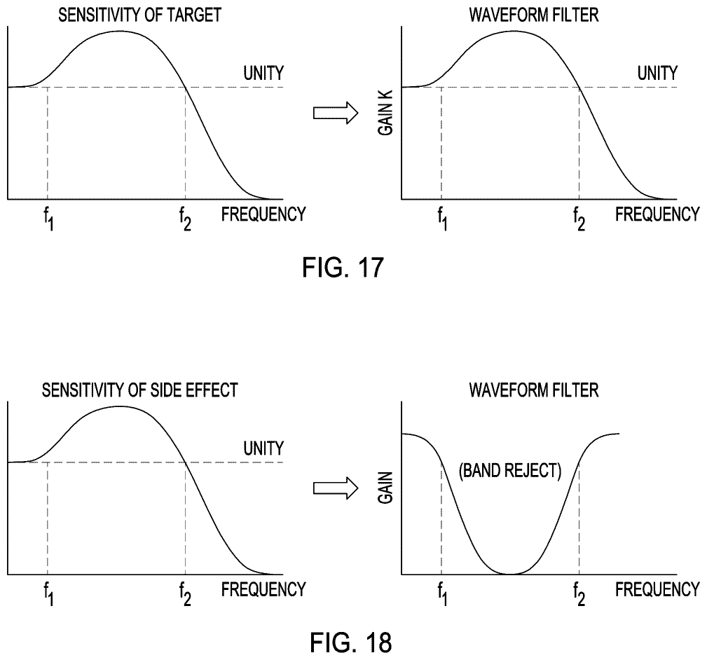

FIG. 17 illustrates, by way of example and not limitation, a sensitivity of a target and a waveform filter that corresponds to the sensitivity of the target. In the illustrated embodiment, the sensitivity of the target is highest between frequencies F1 and F2. The applied waveform filter may apply a gain (K) above unity between the frequencies F1 and F2 to encourage modulation of the target.

FIG. 18 illustrates, by way of example and not limitation, a sensitivity of a side effect and a waveform filter that inversely corresponds to the sensitivity of the side effect. In the illustrated embodiment, the sensitivity of the side effect tissue is highest between frequencies F1 and F2. The applied waveform filter may apply a gain (K) much below unity between the frequencies F1 and F2 to discourage or prevent modulation of the side effect tissue. The applied filter may provide a “band reject” that corresponds to the sensitivity of the side effect tissue.

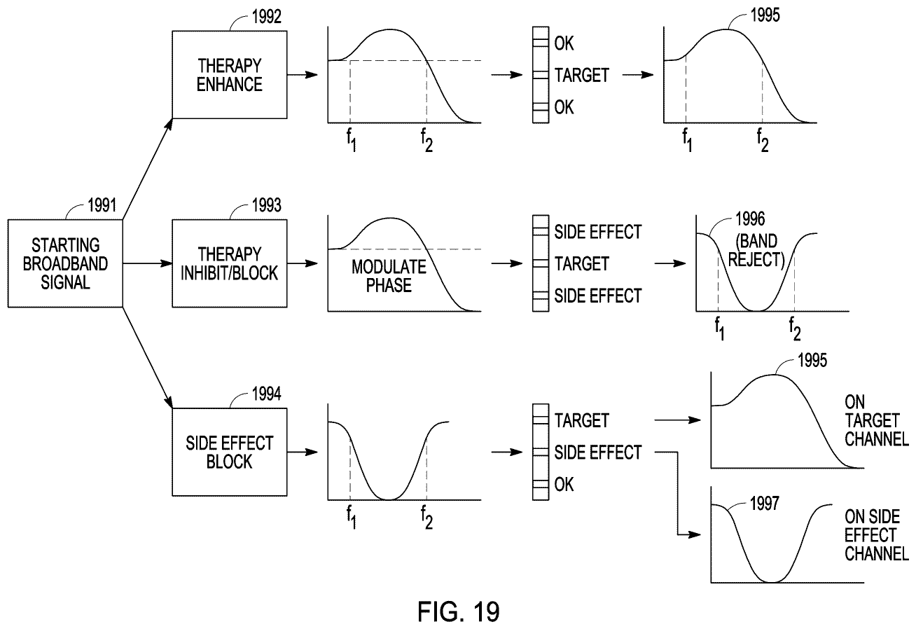

FIG. 19 illustrates, by way of example and not limitation, a decision tree for making spectral adjustments to a broad-spectrum signal 1991 for therapy enhancement 1992, therapy block or inhibition 1993, or side effect block or inhibition 1994. Users may travel down “branches” of the decision tree by answering questions regarding the target/region to be stimulated. Questions may also be tuned according to, by way of example and not limitation, whether the brain, spinal cord, peripheral nerve, and/or a specific body system is being stimulated, which may be queried or automatically set according to therapy-specific information such as the electrode type, patient profile, and the like. In the illustrated embodiments, the starting signal 1991 may be a broad-spectrum signal such as noise (white, Gaussian, 1/f, pink, etc. . . . ), a composite sum of sinusoids, a rectangular pulse pattern, a pulse pattern with one or more parameters modulated by waveform(s), or another waveform pattern that may be nonregular or non-tonic.

Spectral band profiles may describe signal and/or filter through which signal is processed to achieve final result. The user may provide the target characteristics. Electrode settings may be adjusted. The user may be asked about other targets in the area of the electrode span. For example, a therapy enhance decision 1992 may use a filter that applies a gain K between frequencies for which the neural target is sensitive. A final signal/filter type 1995 may be provided for the target characteristics and targets within the electrode span. A therapy inhibit or block decision 1993 may determine the characteristics for modulating side effect target to be blocked, adjust electrode settings for targets within the electrode span, and the provide a final signal/filter type 1996, which is generally an inverse, to inhibit or block at the target(s). A side effect block may provide an inverse gain signal 1997 between frequencies for which the side effect region is sensitive. Electrode settings may be adjusted to account for side effect region(s) and target region(s) within the electrode span. A normal therapy signal or therapy enhance signal may be applied at target(s) while the inverse gain signal 1997 may be applied at side effect region(s).

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
receiving at least a first user input for targeting energy to a volume of tissue, wherein the energy corresponds to a broad-spectrum signal having a plurality of frequency ranges;
determining a stimulation configuration, including the modulation parameter set, based on the first user input; and
delivering the energy corresponding to the broad-spectrum signal using the plurality of electrodes according to the determined stimulation configuration.

2. The method of claim 1, wherein the at least the first user input defines a target type for the volume of tissue and a direction of a modulation field generated by the delivered energy, and the stimulation configuration is automatically determined based on the target type, the direction of the modulation field, or both the target type and the direction of the modulation field.

3. The method of claim 2, further comprising presenting on a user interface for user selection more than one target type or more than one target type proxy corresponding to the more than one target type, wherein the more than one target type includes at least one of an axon/dendrite cable, a cell, a terminal, or another pre-loaded target type.

4. The method of claim 2, further comprising presenting on a user interface a directional axis that can be moved via user interaction to define the direction of the modulation field.

5. The method of claim 1, further comprising presenting on a user interface a target visualization corresponding to a user-selected target type or user-selected target type proxy and imposing a modulation field direction on the target visualization.

6. The method of claim 1, wherein the at least the first user input identifies a location of the volume of neural tissue which corresponds to a target type geometry for the volume of neural tissue, and the stimulation configuration is automatically determined based on the identified location.

7. The method of claim 1, wherein the volume of tissue includes at least one of a targeted volume or a side effect volume, the method further including determining a spatial sensitivity for the at least one of the targeted volume or the side effect volume, wherein the spatial sensitivity represents a proclivity of the volume of tissue to be affected by the delivered energy, and the stimulation configuration is determined using the determined spatial sensitivity.

8. The method of claim 7, wherein the determining the spatial sensitivity includes determining one or more spatial sensitivities for one more targeted volumes and determining one or more spatial sensitivities for one or more side effect volumes.

9. The method of claim 1, further comprising:
receiving at least a second user input for spectrally controlling the broad-spectrum signal;
spectrally adjusting the broad-spectrum signal based on the second user input to provide a spectrally-adjusted signal; and
delivering energy corresponding to the spectrally-adjusted signal using a plurality of electrodes.

10. The method of claim 9, wherein the delivering energy includes using at least a first channel to deliver energy with a first phase to a first tissue volume and a second channel to deliver energy with a second phase to a second tissue volume, and wherein the first and second phases are different.

11. The method of claim 10, wherein the second user input determines a phase for at least one of the first channel or the second channel.

12. The method of claim 10, wherein the delivering energy further includes using at least a third channel to deliver energy with a third phase to a third tissue volume, wherein the first, second and third phases are different.

13. The method of claim 10, wherein the first tissue volume includes a targeted volume of tissue and the second tissue volume includes a side effect volume of tissue, wherein the first and second phases are offset from each other by pi radians such that the energy delivered using the second channel has opposite polarity with respect to the energy delivered using the first channel.

14. The method of claim 10, wherein the energy in the first channel is delivered through a first filter and the energy in the second channel is delivered through a second filter, and the first and second filters are configured to provide the different first and second phases.

15. The method of claim 14, wherein the first and second filters are all-pass filters across the plurality of frequency ranges.

16. The method of claim 14, wherein the first and second filters have different frequency responses with different spectral band profiles across the plurality of frequency ranges.

17. The method of claim 9, wherein the broad-spectrum signal is spectrally-adjusted with a spectral band profile corresponding to a type of tissue volume.

18. The method of claim 1, wherein the broad-spectrum signal includes a waveform pattern modulated by a modulation signal to provide a time varying-pattern, wherein the time varying pattern includes at least one of a dynamic pulse width, a dynamic amplitude or a dynamic frequency, the method further comprising:
receiving feedback; and
adjusting at least one of the waveform pattern or the modulation signal based on the received feedback.

19. The method of claim 1, wherein the broad-spectrum signal is a broad-spectrum digital signal, the method further comprising:
converting the broad-spectrum digital signal into a broad-spectrum analog signal; and
introducing dithering noise into the broad-spectrum analog signal to provide a modified broad-spectrum analog signal,
wherein the delivered energy corresponds to the modified broad-spectrum analog signal using a plurality of electrodes.

20. A non-transitory machine-readable medium including instructions, which when executed by a machine, cause the machine to perform a method comprising:
receiving at least a user input for targeting energy to a volume of tissue, wherein the energy corresponds to a broad-spectrum signal having a plurality of frequency ranges;
determining a stimulation configuration, including the modulation parameter set, based on the user input; and
delivering the energy corresponding to the broad-spectrum signal using the plurality of electrodes according to the determined stimulation configuration.
